(19) United States
(12) Patent Application Publication
(10) Pub. No.: US 2018/0104037 A1
(43) Pub. Date: Apr. 19, 2018

(54) INCONTINENCE GARMENT SYSTEM AND METHOD

(71) Applicant: Eilise Johnson, Charlottesville, VA (US)

(72) Inventor: Eilise Johnson, Charlottesville, VA (US)

(21) Appl. No.: 15/784,050

(22) Filed: Oct. 13, 2017

Related U.S. Application Data
(60) Provisional application No. 62/407,816, filed on Oct. 13, 2016.

(51) Int. Cl. A61F 2/00; A61F 13/496; A41B 9/02; A61F 13/491; A61F 13/49; A61F 13/00; A61F 13/62; A61F 5/44

(52) U.S. Cl. CPC A61F 2/0009; A61F 13/496; A41B 9/026; A61F 13/4915; A61F 13/49011; A61F 2013/8497; A61F 13/622; A61F 13/49006; A61F 5/4401; A61F 2013/49088; A61F 13/00059

(57) ABSTRACT

An incontinence undergarment for adult men. The interior of the undergarment is lined and includes an elastic waistband and elastic leg openings. The undergarment also includes a hook and loop closure strip in the crotch area to enable easy urination without requiring complete removal of the garment. Along the sides of the closure strip are reinforcements, where the interior of the closure strip and the reinforcement sections are lined with plastic to prevent leakage. The undergarment may be available in various sizes, including S, M, L, and XL. It may also be available in a variety of color options.

### CROSS-REFERENCE TO RELATED APPLICATION(S)

The present application is related to and claims priority to U.S. Provisional Patent Application No. 62/407,816 filed Oct. 13, 2016, which is incorporated by reference herein in its entirety.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### TECHNICAL FIELD

The present invention relates generally to the field of garments of existing art and more specifically relates to undergarments.

### RELATED ART

Underwear are items of clothing worn beneath outer clothes, typically in direct contact with the skin. They serve to keep outer garments from being soiled or damaged by bodily excretions, to lessen the friction of outerwear against the skin, to shape the body, and to provide concealment or support for parts of it. There are many men, especially older males that rely on adult diapers for leakage issues and incontinence. Although these diapers are ideal for absorption, they are not designed to feel like conventional underwear. In order to use the restroom while wearing them, the user has to pull them down, which often results in a loss of balance. In an urgent situation, it can be difficult for the individual to remove the undergarment in a timely fashion. In addition, the style can lead some men to feel a loss of confidence. A suitable alternative is needed.

U.S. Pat. No. 8,702,667 to Gwendel Johnson relates to a male incontinence garment. The described male incontinence garment includes a garment which includes an undergarment defining an interior space and an exterior space. An aperture enables the penis of the wearer to extend through the aperture from the interior space to the exterior space. A flap at least partially covers the aperture and is selectively movable between an open position and a closed position. A pouch is located between the flap in the closed position and the undergarment. In a further example, a pad is configured to be placed within the pouch and confine the penis as it extends from the interior space of the undergarment, through the aperture, and into the pad. A method includes placing the undergarment about the body of the male incontinence garment wearer, placing the penis of the male incontinence garment wearer through the aperture and the hole into the pad.

### SUMMARY OF THE INVENTION

In view of the foregoing disadvantages inherent in the known garments art, the present disclosure provides a novel incontinence garment system. The general purpose of the present disclosure, which will be described subsequently in greater detail, is to provide an efficient and effective incontinence garment system.

The present invention offers an absorbent male undergarment that can be used by men with incontinence, leakage issues, or temporary health problems. It provides a ‘fly’ that can be opened to enable men to maintain a sense of normalcy. It eliminates the need to pull the undergarment down to use the bathroom, which can result in a loss of balance. It allows for quick and easy access, ideal for use in an urgent situation. The present invention affords a comfortable, breathable material with great absorption capabilities.

An incontinence garment system is disclosed herein, the incontinence garment system comprising: an incontinence garment having an interior-surface; an exterior-surface; a waistband (having a perimeter configured to fit about a waist of a user-wearer); a first-leg-hole; a second-leg-hole; and a front-panel assembly (having a panel-body; and a closure-strip having fastening means); wherein the incontinence garment system comprises the incontinence garment. The incontinence garment is defined by the interior-surface and the exterior-surface integral with each other. In preferred embodiments the interior-surface is ‘lined’; the incontinence garment formed in certain embodiments as a multi-layer-garment.

The incontinence garment comprises the waistband the first-leg-hole, the second-leg-hole in functional combination such that the incontinence garment is able to form-fit about the user-wearer for wear-use and yet fold substantially flat during non-use periods. The waistband, the first-leg-hole, the second-leg-hole each preferably comprise elastic such that they are each independently elastically-deformable. The front-panel assembly comprises the panel-body and the closure-strip in functional combination able to act in a capacity of a fly (which is able to be fastened in a closed-condition via the fastening means when desired; the closure-strip able to be uncoupled from a coupled relationship with the panel-body when desired in an opened-condition). The penis on the user-wearer is able to be pulled out of an aperture in the panel-body to provide for unobstructed urination and the penis reinserted within the confined of the incontinence garment after urination is completed.

The closure-strip is able to be rotated in relation to the panel-body to allow repeated movement between the closed-condition and the opened-condition, as desired for non-urination instances and urination instances. The present invention is also designed for ease of use by those with poor dexterity. The fastening means of the closure-strip comprises hook and loop fasteners in preferred embodiments and may comprise alternate fastening means in other embodiments. Examples of other suitable equivalents may comprise re-useable adhesive means, buttons, snaps, zipping means, tongue and groove (press seal) such as those used on ZIPLOCK® and the like.

The closure-strip comprises reinforcing means. The interior-side of the closure-strip is preferably lined with plastic (or other suitably equivalent material) to prevent leakage when the closure-strip is coupled to the panel-body. The fly is vertically-set when the incontinence garment is worn and is configured such that use of the fly provides that the incontinence garment does not need to be pulled down for urination-use. Further, since it swings along the vertical axis it is not prone to ‘drippage’ of urine onto the ‘fly’ during and after urination thus providing a garment that is easier to keep clean; the fly does not open downwardly In preferred embodiments the incontinence garment comprises fabric; wherein the fabric is breathable and comprises a weave. As such, the incontinence garment is configured to provide absorbent-garment that can be used by men with incontinence, and leakage issues. According to the embodiment, a method of use is also disclosed herein.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, an incontinence garment system, constructed and operative according to the teachings of the present disclosure.

FIG. 1 is a perspective view of the incontinence garment system during an ‘in-use’ condition, with a user opening the front-panel assembly for urination according to an embodiment of the disclosure.

FIG. 2 is a perspective view of the incontinence garment demonstrating how the hook and loop closure tab (of the closure-strip) is able to be opened of FIG. 1, according to an embodiment of the present disclosure.

FIG. 3 is a perspective view of the incontinence garment of the incontinence garment system with the fly closed of FIG. 1, according to an embodiment of the present disclosure.

FIG. 4 is an interior view of the incontinence garment of FIG. 1, according to an embodiment of the present disclosure.

FIG. 5 is a perspective view of the incontinence garment of FIG. 1, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to an undergarment and more particularly to an incontinence garment system as used to improve the efficiency, utility and comfort of undergarments.

Generally, ‘Open Fly Undergarment’ (incontinence garment system) provides an incontinence undergarment for adult men. The interior of the undergarment is lined and includes an elastic waistband and elastic leg openings. The undergarment also includes a hook and loop closure strip in the crotch area to enable easy urination without requiring complete removal of the garment. Along the sides of the closure strip are reinforcements, where the interior of the closure strip and the reinforcement sections are lined with plastic to prevent leakage. The undergarment may be available in various sizes, including S, M, L, and XL. It may also be available in a variety of color options.

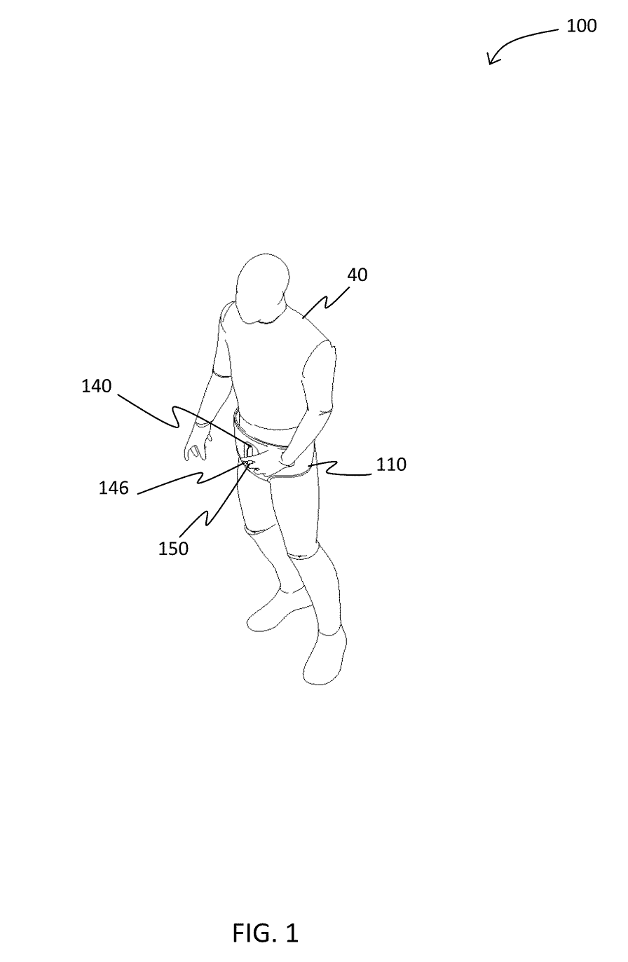

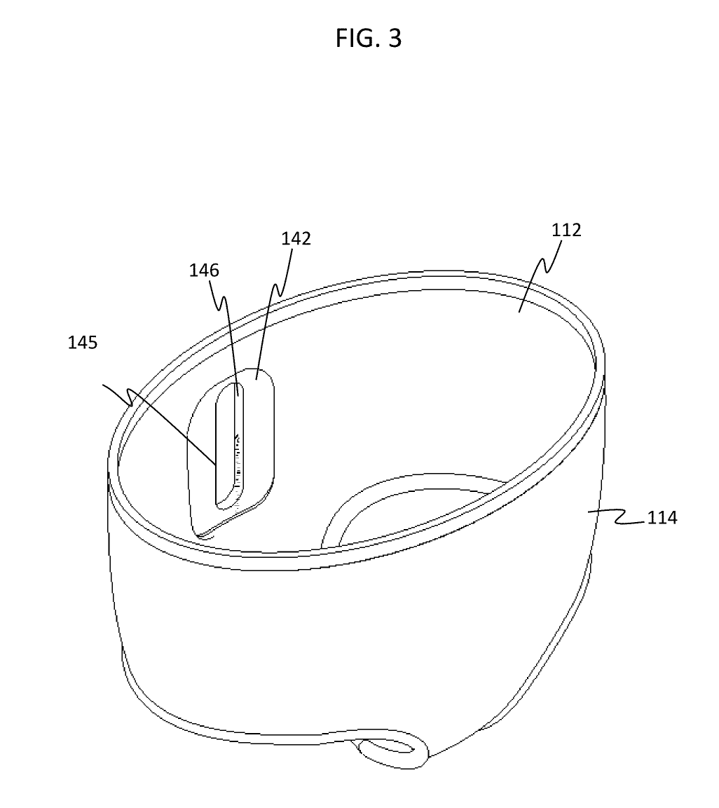

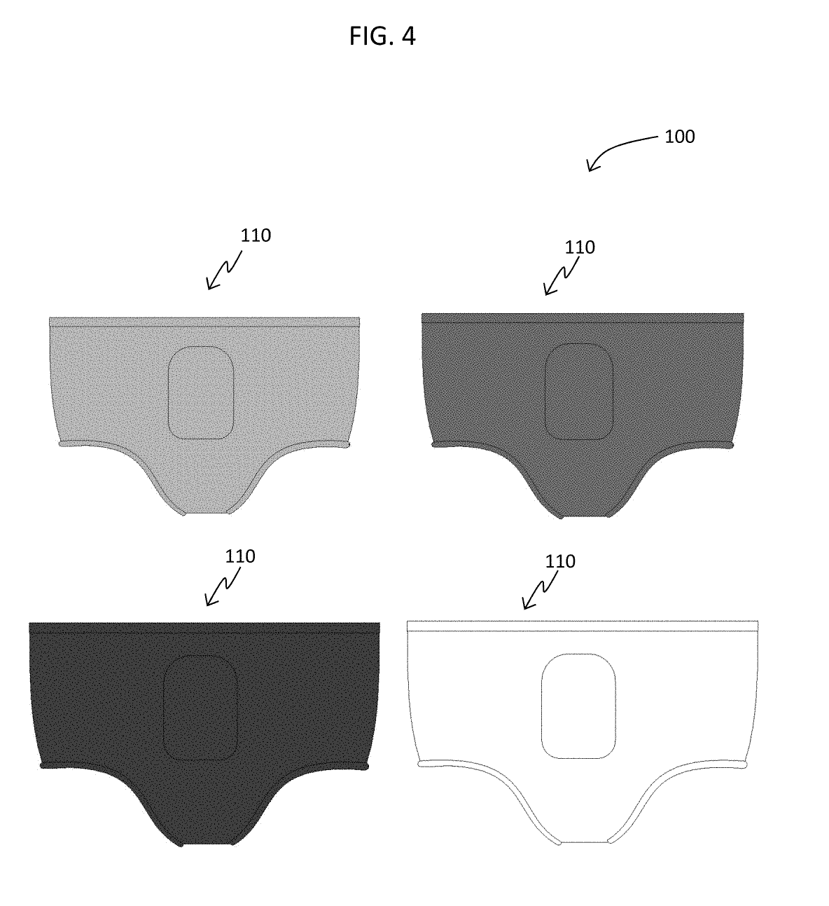

Referring now more specifically to the drawings by numerals of reference, there is shown in FIGS. 1-5, various views of an incontinence garment system 100.

FIG. 1 shows an incontinence garment system 100 during an ‘in-use’ condition 50, according to an embodiment of the present disclosure. Here, the incontinence garment system 100 may be beneficial for use by a user-wearer 40 to provide comfortable undergarments suitable for use by incontinent user-wearers 40. As illustrated, the incontinence garment system 100 may include an incontinence garment 110 having an interior-surface 112; an exterior-surface 114; a waistband 120 (having a perimeter configured to fit about a waist of a user-wearer 40); a first-leg-hole 130; a second-leg-hole 132; a front-panel assembly 140 (having a front-mounted panel-body 142 and a closure-strip 144 having fastening means 146); wherein the incontinence garment system 100 comprises the incontinence garment 110; the incontinence garment 110 defined by the interior-surface 112; and exterior-surface 114 integral with each other. As designed, the incontinence garment 110 comprises the waistband 120, the first-leg-hole 130; and second-leg-hole 132 in functional combination such that the incontinence garment 110 is able to form-fit about the user-wearer 40 for wear-use.

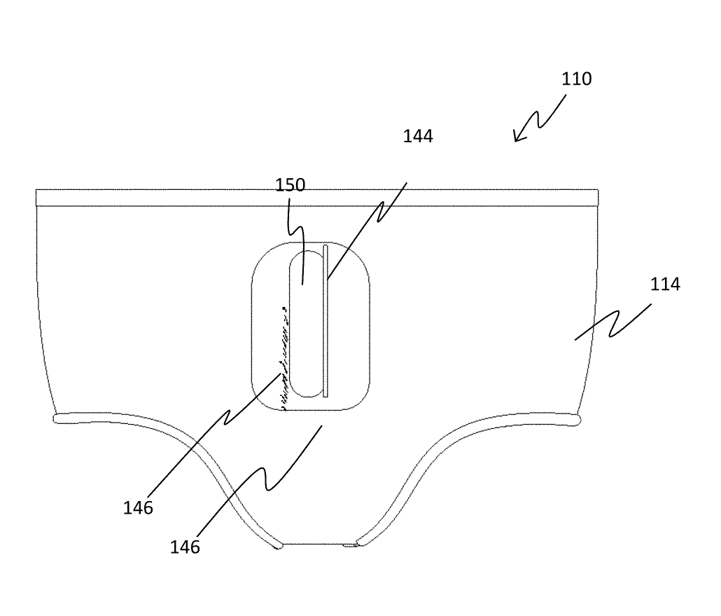

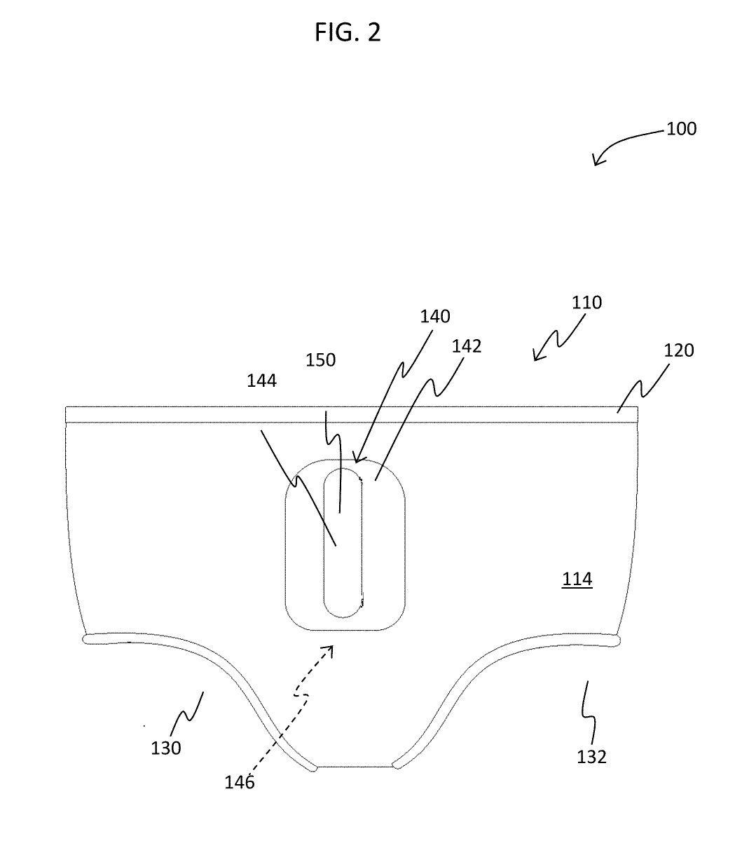

Referring now to the front-panel assembly 140; the front-panel assembly 140 comprises the panel-body 142 and the closure-strip 144 in functional combination able to act in a capacity of a ‘fly’ 150 which is able to be fastened in a closed-condition (shown in FIG. 3) via the fastening means 146 when desired, the closure-strip 144 able to be uncoupled from a coupled relationship with the panel-body 142 when desired in an opened-condition. As designed, the closure-strip is able to be rotated (about a vertical axis) in relation to the panel-body to allow repeated movement between the closed-condition and the opened-condition, as desired (shown in FIGS. 1-2). The interior-surface 112 is lined, the incontinence garment 110 formed as a multi-layer-garment. The incontinence garment 110 is configured to provide an absorbent-garment that can be used by men with incontinence, and with leakage issues. The present invention may be worn by user(s) 40 where other health issues may be present.

The waistband 120 comprises elastic or other suitable deforming means. The first-leg-hole 130 and the second-leg-hole 132 also comprise elastic. The incontinence garment 110 may come in a plurality of colors; wherein the plurality of colors provide functional aesthetic means for use in matching to outer garments, may be used for ready identification (day of the week or the like). The incontinence garment 110 is able to be produced in small, medium, large sizes.

Referring now to the fastening means 146 of the closure-strip 144, in preferred embodiments the fastening means 146 used comprises hook and loop fasteners such as that sold under the tradename VELCRO®. Other suitable fastening means may be used. The fly 150 is vertically-set when the incontinence garment 110 is worn; wherein the incontinence garment 110 is configured such that use of the fly 150 provides that the incontinence garment 110 does not need to be pulled down for urination-use. Those with ordinary skill in the art will now appreciate that upon reading this specification and by their understanding the art of fastening as described herein, methods of fastening will be understood by those knowledgeable in such art.

The closure-strip 144 comprises reinforcing means and an interior-side 145 of the closure-strip 144 is also preferably lined with plastic to prevent leakage when the closure-strip 144 is coupled to the panel-body 142. The incontinence garment 110 preferably comprises fabric; wherein the fabric is breathable since it comprises a weave. Upon reading this specification, it should be appreciated that, under appropriate circumstances, considering such issues as user preferences, design preference, structural requirements, marketing preferences, cost, available materials, technological advances, etc., other material arrangements such as, for example, use of weaved and un-weaved manufactures, use of materials other than fabric, materials such as absorbent materials, use of other sealing means, non-absorbent portions, porous materials, composites, etc., may be sufficient.

According to one embodiment, the incontinence garment system 100 may be arranged as a kit 105. In particular, the incontinence garment system 100 may further include a set of instructions 107. The instructions 107 may detail functional relationships in relation to the structure of the incontinence garment system 100 such that the incontinence garment system 100 can be used, maintained, or the like, in a preferred manner. The kit 105 may come with a plurality of incontinence garments 110 offered in a plurality of colors, as previously described; wherein the plurality of colors provide functional aesthetic means for use in matching to outer garments, may be used for ready identification (day of the week or the like).

A method for using the incontinence garment system 100, according to an embodiment of the present disclosure may include one or more components or features of the incontinence garment system 100 as described above. As illustrated, the method for use for the incontinence garment system 100 may include the steps of: providing the incontinence garment 110 as described herein; donning the incontinence garment 110; opening the closure-strip 144 in relation to the panel-body 142 (located on the front of the incontinence garment 110 when worn) to urinate; fastenably-closing (sealing) the closure-strip 144 in relation to the panel-body 142 (such that it provides a ‘sealed-condition). The incontinence garment 110 is able to be pulled down for defecating or for removal for changing out to new pair of incontinence garments 110.

It should also be noted that the steps described in the method of use can be carried out in many different orders according to user preference. The use of “step of” should not be interpreted as “step for”, in the claims herein and is not intended to invoke the provisions of 35 U.S.C. §112(f). It should also be noted that, under appropriate circumstances, considering such issues as design preference, user preferences, marketing preferences, cost, structural requirements, available materials, technological advances, etc., other methods for providing incontinence protections means are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. An incontinence garment system, the incontinence garment system comprising:
an incontinence garment having;
an interior-surface;
an exterior-surface;
a waistband having a perimeter configured to fit about a waist of a user-wearer;
a first-leg-hole;
a second-leg-hole; and
a front-panel assembly having;
a panel-body; and
a closure-strip having fastening means;
wherein said incontinence garment system comprises said incontinence garment, said incontinence garment defined by said interior-surface and said exterior-surface integral with each other;
wherein said incontinence garment comprises said waistband, said first-leg-hole, and said second-leg-hole in functional combination such that said incontinence garment is able to form-fit about said user-wearer for wear-use;
wherein said front-panel assembly comprises said panel-body and said closure-strip in functional combination able to act in a capacity of a fly which is able to be fastened in a closed-condition via said fastening means when desired, the closure-strip able to be uncoupled from a coupled relationship with said panel-body when desired in an opened-condition;
wherein said closure-strip is able to be rotated in relation to said panel-body to allow repeated movement between said closed-condition and said opened-condition, as desired; and
wherein said incontinence garment is configured to provide absorbent-garment that can be used by men with incontinence, and leakage issues.

2. The incontinence garment system of claim 1, wherein said interior-surface is lined, said incontinence garment formed as a multi-layer-garment.

3. The incontinence garment system of claim 1, wherein said waistband comprises elastic.

4. The incontinence garment system of claim 1, wherein said first-leg-hole comprises elastic.

5. The incontinence garment system of claim 1, wherein the second-leg-hole comprises elastic.

6. The incontinence garment system of claim 1, wherein the incontinence garment comes in a plurality of colors.

7. The incontinence garment system of claim 6, wherein the plurality of colors provide functional aesthetic means for identification.

8. The incontinence garment system of claim 1, wherein the fastening means of the closure-strip comprises hook and loop fasteners.

9. The incontinence garment system of claim 1, wherein the fly is vertically-set when said incontinence garment is worn.

10. The incontinence garment system of claim 9, wherein the incontinence garment is configured such that use of the fly provides that the incontinence garment does not need to be pulled down for urination-use.

11. The incontinence garment system of claim 1, wherein the closure-strip comprises reinforcing means.

12. The incontinence garment system of claim 1, wherein an interior-side of the closure-strip are lined with plastic to prevent leakage when closure-strip is coupled to said panel-body.

13. The incontinence garment system of claim 1, wherein the incontinence garment comprises fabric.

14. The incontinence garment system of claim 13, wherein the fabric is breathable.

15. The incontinence garment system of claim 1, wherein the fabric comprises a weave.

16. The incontinence garment system of claim 1, wherein the incontinence garment is able to be produced in at least small, medium, large sizes.

17. An incontinence garment system, the incontinence garment system comprising:
an incontinence garment having;
an interior-surface;
an exterior-surface;
a waistband having a perimeter configured to fit about a waist of a user-wearer;
a first-leg-hole;
a second-leg-hole; and
a front-panel assembly having;
a panel-body;
an aperture from which a penis may extend for urination; and
a closure-strip having fastening means;
wherein said incontinence garment system comprises said incontinence garment, said incontinence garment defined by said interior-surface and said exterior-surface integral with each other;
wherein said interior-surface is lined, said incontinence garment formed as a multi-layer-garment;
wherein said incontinence garment comprises said waistband said first-leg-hole, said second-leg-hole in functional combination such that said incontinence garment is able to form-fit about said user-wearer for wear-use;
wherein said waistband, said first-leg-hole, said second-leg-hole each comprises elastic such that they are each independently elastically-deformable;
wherein said front-panel assembly comprises said panel-body and said closure-strip in functional combination able to act in a capacity of a fly which is able to be fastened in a closed-condition via said fastening means when desired, the closure-strip able to be uncoupled from a coupled relationship with said panel-body when desired in an opened-condition for use in urination;
wherein said closure-strip is able to be rotated in relation to said panel-body to allow repeated movement between said closed-condition and said opened-condition, as desired;
wherein the fastening means of the closure-strip comprises hook and loop fasteners;
wherein the closure-strip comprises reinforcing means;
wherein an interior-side of the closure-strip is lined with plastic to prevent leakage when said closure-strip is coupled to said panel-body;
wherein the fly is vertically-set when said incontinence garment is worn;
wherein the fly does not open downwardly;
wherein the incontinence garment is configured such that use of the fly provides that the incontinence garment does not need to be pulled down for urination-use;
wherein the incontinence garment comprises fabric;
wherein the fabric is breathable;
wherein the fabric comprises a weave; and
wherein said incontinence garment is configured to provide absorbent-garment that can be used by men with incontinence, and leakage issues.

18. The incontinence garment system of claim 17, further comprising set of instructions; and
wherein the incontinence garment system is arranged as a kit.
